(19) United States
(12) Patent Application Publication
(10) Pub. No.: US 2005/0148870 A1
(43) Pub. Date: Jul. 7, 2005

(54) Apparatus for generating command signals to an electronic device

(76) Inventors: Henrik Dryselius, Bromma (SE); Staffan Dryselius, Uppsala (SE); Torbjorn Nilsson, Bromma (SE)

Correspondence Address: JACOBSON HOLMAN PLLC, 400 SEVENTH STREET N.W., SUITE 600, WASHINGTON, DC, 20004, US

(21) Appl. No.: 10/513,328

(22) PCT Filed: May 9, 2003
(86) PCT No.: PCT/SE03/00747

(30) Foreign Application Priority Data
May 10, 2002 (SE) 0201434-8

(51) Int. Cl. A61B 8/00

(52) U.S. Cl. 600/437

(57) ABSTRACT

The invention consists of a device to give command signals to an electronic device characterised by being comprised of at least one acoustic sensor (2a and 2b), to be brought in contact with one or more extremities of a user by means of one or more attachments (1a and 1b), for detecting signals generated in the tissues of the user's extremities as these move, the sensor(s) (2a and 2b) being arranged to communicate with at least one signal filtering device (13a and 13b) set up to communicate with the electronic device.

### FIELD OF THE INVENTION

The present invention relates to a device and a method for entering characters and to give command signals to electronic devices such as e.g. computers and mobile phones.

### BACKGROUND OF THE INVENTION

Mobile electronic equipment, especially handheld devices such as e.g. mobile phones and PDA:s (Personal Digital Assistants), are developed into increasingly smaller units. Concurrently, the functions of these devices become more complex and demand ever-larger inputs of information, so that the need for functional keyboards is now a size-limiting factor.

To improve information entry, several alternatives to keyboards have been developed. Examples include devices and methods that register finger movements and uses optical sensors, pressure transducers or ultra sound imaging of the tendons in the wrist. Other approaches record the movements of the fingers by measuring angles and distances of the fingers relative to a keyboard plan that is projected on a flat surface. Also voice analysis, writing on a pressure sensitive surface or the use of a special pen to digitise handwriting have been developed as alternatives to traditional keyboards. All these respective methods have disadvantages and the equipment used is either expensive or is not sufficiently user friendly.

Consequently there is a need for an improved device and method for entering characters and to issue command signals to the type of devices exemplified above.

### DISCLOSURE OF THE INVENTION

The object of the present invention is to achieve an improved and more user friendly technique for entering characters and to issue command signals to electronic devices, e.g. desktop computers, portable computers, handheld computers, mobile phones and other home electronic equipment.

The object is achieved by providing a tool for commanding electronic equipment for detecting signals generated in the tissues during movement. The tool comprises at least one sensor intended to be brought close to at least one extremity of a user by means of one or more attachments. The sensor is arranged in a way so as to communicate with at least one signal-filtering device, which in turn is arranged for communicating with the electronic equipment.

By using the invention, entering characters and giving other types of command signals to electronic devices is independent of the general posture of the user and that of the hands, which leads to ergonomic advantages. The invention does not require a planar surface in front of the electronic device to type on, but gives the user freedom to move around when the device is applied to either or both wrists/forearms. Furthermore, the unobtrusiveness of the device enables the user to use the hands for other tasks, even while typing or giving other command signals.

The device and the methods for its use can be configured to suit individual user needs, in particular to provide solutions for people with orthopaedic impairment or other types of disabilities.

The locution “electronic device”, as used in this document, characterises all possible kinds of electronic equipment of the type exemplified above, where user input of characters and other command signals are required. With characters and other command signals are meant all possible signals denoting single or combined finger movements and all other signals representing letters, other characters, their combinations, or any other type of command signal. Examples of characters and command signals are those sent from keyboards and keypads attached to or integrated in electronic devices. These command signals can be e.g. “a”, “2” and “return”, but also signals representing other types of input devices, such as mouse pointers, remote controls for television sets or commands for portable music players such as “play” and “fast forward” or commands for making a call from a mobile phone.

Input of characters or command signals, represented by finger movements, is detected and identified based on the correlation of the acoustic signals that arise when moving the fingers and previously recorded amplitude and/or frequency patterns (below called signal characteristics).

The acoustic signals are detected, transformed and transferred through a transducer into a memory unit arranged to communicate with a processor that makes the correlation analyses. To enter and save signal characteristics into the memory, the user makes distinct movements with his fingers during a configuration procedure.

### CONCISE DESCRIPTION OF THE DRAWINGS

The invention is described in more detail below with reference to the FIGS. 1 and 2 in which:

FIG. 1 is a schematic drawing of an embodiment of the invention where the device is applied to both forearms/wrists. FIG. 2 shows another embodiment of the invention.

### DETAILED DESCRIPTION OF THE INVENTION

The device comprises one or more sensors, arranged to detect acoustic signals emanating from the user's hand(s) or forearm(s) when moving his hand(s) and finger(s). These movements specify the characters or command signals to be entered into electronic equipment.

Each finger of the hand has tendons that extend through the wrist and attach to muscles in the forearm. When fingers are moved, their joints and especially the tendons of the hand emit sound waves: Rubbing or tapping the fingers also produces sounds emanating from the contact point of the skin. The sound-waves are altered and attenuated as they travel through the tissues, and by appropriate placement of one or more sensors, these sounds can be analysed with respect to which fingers-are moving, and how they move. This information is ultimately used to determine which characters or command signals are entered into electronic devices.

In order to reliably detect the sounds from e.g. the finger tendons, at least one sensor is used, but to facilitate the analysis, preferably as many as five or more sensors can be used to unequivocally determine simultaneous movements of more than one finger. When using less than five sensors (one for each finger), it is more difficult to distinguish between movements by fingers that are used simultaneously, but in principle it is possible to use differences in the signal profile that reaches the sensor(s) from the respective finger movements. When analysing the time-dependent variations in frequency and/or amplitude, based on knowledge of the anatomical placement of the tendons and that of the sensor(s), it is possible to determine what particular finger(s) are moving. By using several sensors placed according to the anatomical positions of the tendons, patterns between the different sensors can also be utilised to facilitate the analysis. One advantage of using more than five sensors is that the attachment organ for the sensors does not have to be applied with the same degree of precision to the forearm/wrist or the back of the hand (dorsal part of the hand). With a sufficient number of sensors, some will always be within adequate range of the tendons to give appropriate signals to determine the movements of the fingers.

In a preferred embodiment of the invention, acoustic sensors are attached to, or integrated in, a bracelet worn on the forearm or wrist. The sensors are placed on the skin, above the tendons that run from their respective fingers through the wrist to the muscles in the forearm. Of course, instead of bracelets, alternative attachments for the sensors are possible.

The detection of acoustic signals does not necessarily imply that the sensors must be in direct contact with the skin, only that they are arranged in a way so as to pick up the sounds from the movements of the fingers. Hence, the invention includes technical solutions where sensors pick up sounds from a distance, through air or other media or where sensors optically register acoustic signals off the surface of the skin.

By placing at least one out of a suitable number of acoustic sensors around e.g. the user's forearm(s)/wrist(s), it is possible to detect single or combined finger movements based on acoustic signals from his finger tendons. The use of more than five sensors will give a wider spectrum of distinguishable signal patterns, also between the sensors, and will make the bracelet less sensitive to exact placement and to anatomical differences between different users.

One or more bracelets can be used (preferably two), on one or more extremities, each with acoustic sensors devised to pick up sounds from wrists/forearms or ankles/lower parts of the legs.

The acoustic sensors can use piezoelectric materials, but in accordance with the invention a wide spectrum of different acoustic sensor technologies can be used.

In another embodiment of the invention, acoustic sensors are placed on the dorsal side of the hand to pick up signals in accordance with the above.

In another embodiment of the invention, acoustic sensors are placed on the knuckles of the hand or are arranged in a way so as to pick up sounds from other joints of the fingers as the fingers move.

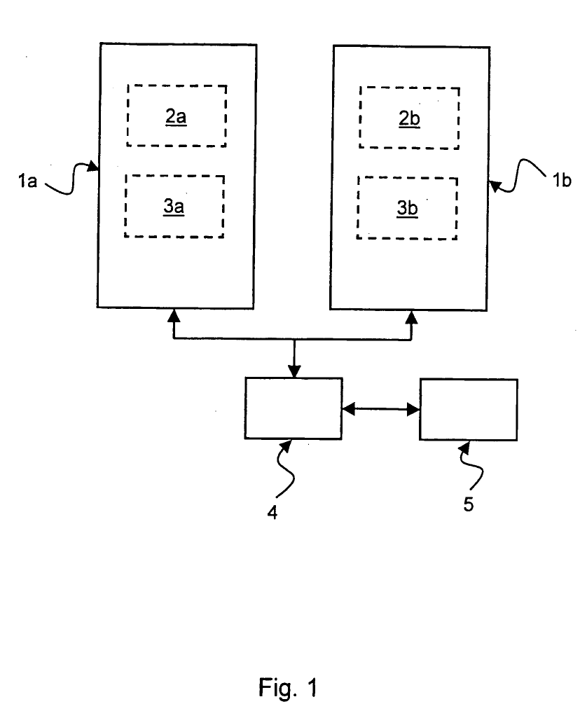

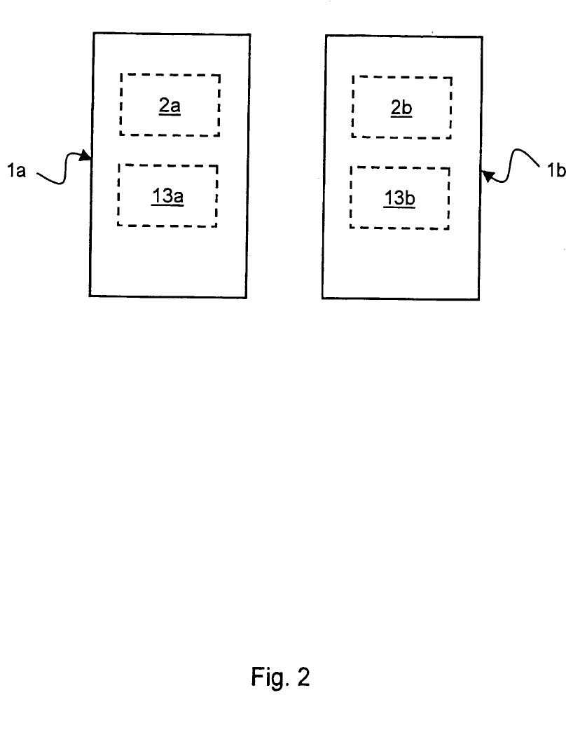

FIG. 1 illustrates an embodiment of the invention comprising the attachment organs 1a and 1b to be placed on a user's wrist(s)/forearm(s). Acoustic sensors 2a and 2b, in this case made from sectioned piezoelectric film, are integrated in a bracelet and brought into contact with the skin. Moving the fingers, acoustic signals mainly originating from the finger tendons and surrounding tissue, are transformed into electrical signals by the sensors 2a and 2b.

The signals are adapted and reshaped in the transducer units (3a and 3b), here including digitising by A/D converters, before they are transmitted by wire or wirelessly to a signal processing unit (4), including a processor, where they are analysed. The processor unit can be an integral part of an electronic device to which signals are entered, or a separate unit with means for communication. The communication can be achieved with e.g. small receivers and transmitter modules for radio frequencies adhering to e.g. the Bluetooth standard that uses the free ISM frequency at approximately 2.4 GHz. Also with the bracelet(s), e.g. Bluetooth modules should preferably be used to communicate with the processor unit. The processor unit then relays analysed signals for different characters or other command signals to the electronic device. Of course all or parts of the communication can use other techniques, such as e.g. cable or infrared link.

Since the acoustic signals are attenuated as they travel through the tissues of the hand and/or arm, the sensors closest to the origin of the sound will pick up the strongest signals. In the processor unit 4, the signals from the sensors 2a and 2b are analysed with respect to amplitude and/or frequency patterns to determine what particular fingers are moving, and finally to determine which character or command signal the user intends to make. The analysis is based on a comparison of the signal from the sensors and on the reference patterns saved in the memory unit 5, which is set up to communicate with the processor unit 4. The reference patterns are initially saved into the memory unit during the configuration process, during which the movements of the fingers are linked to the respective signal characteristics.

Letters and characters represented by different finger movements can be determined from standard keyboard plans, e.g. QWERTY (named after the first six keys), or from single or combined finger movements, each representing characters or other command signals.

The analysis of the meaning of single or combined finger movements can be done in the electronic device, in which case only information about which finger is moving is sent to it, or in the processor 4. Analysing keystrokes based on a keyboard plan (QWERTY or other), uses the fact that each finger only presses a limited number of keys. Once a series of finger movements is followed by a spacebar stroke (thumb movement), a word has been written, and its correct meaning can be determined by matching the signals against possible combinations of letters and characters and grammatical rules which have been saved previously (pattern matching).

Chord typing, to enter characters using combinations of finger movements, is an alternative or complementary input technique. Each character or command signal is given a unique combination of movements by one or more fingers. Since the alphabet is sequential, there are several possible alternatives for chord typing that can be easily memorised. It is also conceivable to assign certain finger movements a small number of figurative symbols that can be combined to form Western alphabetic letters or symbols, or e.g. Chinese or Japanese ones, or those of any other alphabet e.g. the Cyrillic alphabet.

The start of the input of a character or command signal can be discerned by the first -part of the movement of one or more fingers, and the end of the input sequence is marked by its return to the original position.

The invention is not limited to replacing keystrokes; it can also emulate e.g. mouse-input signals.

The sound from rubbing e.g. the index or middle finger against the thumb on one hand can be used to move the mouse pointer in e.g. the horizontal direction. To move in the orthogonal direction, the corresponding fingers of the other hand can be rubbed. Of course, the method can be generalised to more dimensions. The emulation of mouse movements can be made as a combination of the sound from the finger tendons and the friction from the sliding contact point between the thumb and other fingers, the sound of which travels mainly through the bones of the hands to the sensors. In a similar way, left and right mouse clicks can be defined as e.g. the sound from tapping e.g. the index or middle finger against the thumb.

In an alternative or complementary embodiment (not shown), a touch pad or a touch screen, preferably integrated in one of the attachment organs for the sensors, can be set up to communicate with the processor.

In alternative or complementary embodiments, the invention is equipped with presentation means (not shown). These can be arranged both to provide feedback to the entering of characters and other command signals and as output units for the processor or connected electronic devices. The means of presentation can be integrated in at least one of the attachment organs for the sensors, or it can be a separate unit or it can be arranged in some other suitable way. Especially when the means of presentation is a display or one or more vibrators, and the attachment organ is a bracelet, its integration in the attachment organ is favoured. Using acoustic means of presentation, e.g. an earphone (possibly with wireless means of communication), can be favourable. Feedback to the user can be simple sound signals denoting different kinds of input, or synthetic speech reflecting the input of characters, the combinations of these or other command signals into an electronic device.

In FIG. 2 another embodiment of the invention is depicted, comprising attachment organs 1a and 1b for application on a user's forearms/wrists. Acoustic sensors 2a and 2b are adjusted to substantially contact the skin, in this case piezoelectric sensors embedded in bracelets. Moving the fingers, acoustic signals are produced mainly from the finger tendons moving against the surrounding tissue. The acoustic sensors, five in this embodiment, are placed immediately above the respective finger tendons on the lower (volar) side of the wrist/forearm. The acoustic signals are transduced into electric signals by the sensors 2a and 2b and processed by a filter unit 13a and 13b, which discriminates and digitises signals in a predefined amplitude interval. The signals are relayed to the electronic device using e.g. a Bluetooth module integrated in the bracelet. In this embodiment, the amplitude alone is sufficient to indicate what particular finger(s) are moving. No matching of the signals against a saved pattern is required, either between responses from the different sensors or in the form of special frequency content. Analysis of the meaning of the finger movements is performed in the electronic device. The filter device 13a and 13b can also be integrated in the electronic device, in which case it receives unprocessed signals from the sensors 2a and 2b.

The invention is not limited to the embodiments outlined above, but may be modified according to the enclosed claims of the invention.

1. A device to give command signals to an electronic device characterised by being comprised of at least one acoustic sensor (2a), to be brought in contact with one or more extremities by means of one or more attachments (1a), for detecting signals generated in the tissues of the user's extremities as the extremities move, the sensor (2a) being arranged to communicate with a processor unit (4), and the processor unit (4) being arranged to communicate with a memory (5) in which at least one signal characteristic comprising frequency and/or amplitude data is saved, the processor unit (4) being arranged to analyse the signals detected by the sensor (2a) with respect to the correlation with the signal characteristic, in which case a substantial positive correlation results in the generation of the above mentioned command signals.

2. A device to give command signals to an electronic device characterised by being comprised of at least one acoustic sensor (2a), to be brought in contact with one or more extremities of a user by means of one or more attachments (1a), for detecting signals generated in the tissues of the user's extremities as these move, the sensor (2a) being arranged to communicate with at least one signal filtering device (13a) set up to communicate with the electronic device.

3. A device according to claim 1 characterised by the command signals representing single or combined finger movements.

4. A device according to claim 1 characterised by the command signals representing the same types of signals as those sent by other types of input devices used in conjunction with electronic devices.

5. A device according to claim 1 characterised by the sensor (2a) being made of piezoelectric material.

6. A device according to claim 5 characterised by the sensor being made of a sectioned piezoelectric film.

7. A device according to claim 1 characterised by the attachment (1a) being a bracelet arranged so that acoustic signals originating in the user's extremities can be detected with the sensor (2a).

8. A device according to claim 1 characterised by the movements to be detected being hand-, finger-or toe movements.

9. A device according to claim 1 characterised by the sensor being arranged to detect acoustic signals originating from movements of the tendons embedded in the tissues of the extremities.

10. A device according to claim 1 characterised by the sensor (2a) being arranged to detect acoustic signals originating from movements of the muscles embedded in the tissues of the extremities.

11. A device according to claim 1 characterised by the sensor (2a) being arranged to detect acoustic signals originating in the joints of the hands of a user.

12. A device according to claim 1 characterised by the means of presentation being arranged for both feedback to the user and as output interface for the processor unit 4 or connected electronic devices.
